(19) United States
(12) Patent Application Publication
(10) Pub. No.: US 2003/0144582 A1
(43) Pub. Date: Jul. 31, 2003

(54) Portable non-invasive glucose monitor

(76) Inventors: Carl Cohen, Newton, MA (US); John Hurrell, Carmel, IN (US)

Correspondence Address: HELLER EHRMAN WHITE & MCAULIFFE LLP, SUITE 300, 101 ORCHARD RIDGE DR., GAITHERSBURG, MD, 20878-1917, US

(21) Appl. No.: 10/237,193

(22) Filed: Sep. 9, 2002

Related U.S. Application Data
(60) Provisional application No. 60/317,436, filed on Sep. 7, 2001.
(60) Provisional application No. 60/317,484, filed on Sep. 7, 2001.

(51) Int. Cl. A61B 5/00

(52) U.S. Cl. 600/316

(57) ABSTRACT

The invention provides compositions, methods and devices for noninvasive measurement of the analyte levels in vivo. More specifically, the invention provides a hand-held glucose monitor and an optical coupler that allows for short-term discontinuous and/or continuous information on dynamic in vivo glucose levels. The device may include a optical coupler for optically connecting a skin surface to the device that contains a plurality of zones. These zones contain areas for a variety of purposes including calibration of the instrument, reading of the skin surface, and protection of the instrument.

### FIELD OF THE INVENTION

The invention provides to devices, compositions and methods for determining the concentration of one or more analytes in a biological sample. In particular, the invention provides devices, compositions and methods for the determination of in vivo glucose levels.

### BACKGROUND OF THE INVENTION

The ability to regulate and maintain a stable physiologic environment is a key differentiating aspects between health and illness. Loss of the ability to regulate carbohydrate, fat, and protein metabolism characterizes a disease known as diabetes mellitus. At least three major subgroups of this disorder have been identified and in all three the loss of normal carbohydrate regulatory mechanisms puts disease sufferers at risk for complications of secondary to intermittent hyperglycemia.

Type I diabetes accounts for about 10% of diabetics and is characterized by a severe insulin deficiency resulting from a loss of insulin-secreting beta cells in the pancreas. The remaining 90% of diabetic patients suffer from Type 2 diabetes, which is characterized by insulin resistance or an impaired insulin response in the peripheral tissues (Robbins, S. L. et al., Pathologic Basis of Disease, 3rd Edition, W. B. Saunders Company, Philadelphia, 1984, p. 972). When insulin production is reduced or insulin receptor sensitivity is decreased, normal glucose transport into cells is disrupted. Untreated, this results in elevated levels of blood glucose, or hyperglycemia, which remains the most frequent characterization of diabetes.

The human body is optimized to function when the blood glucose levels range between 80-100 mg/dl. Most tissues can use fatty acids as their primary if not sole source of metabolic energy. However, there are notable exceptions to this general rule. Brain and other nervous tissues employ glucose as an obligate energy source. Red blood cells, since they do not contain mitochondria, can obtain energy only by anaerobic glycolysis. Skeletal muscle at rest uses predominantly lipid as the energy source but in heavy exercise also draws upon muscle glycogen and blood glucose.

Because brain and red blood cells depend almost exclusively upon glucose as their source of energy, it is essential that it always be available. Insofar as free glucose is present in the plasma and interstitial fluid at a concentration of approximately 80 mg per 100 ml, a typical 70 kg person has about 20 grams of free glucose. Approximately 180 grams of glucose are oxidized per day, mostly by those tissues for which it is essential. The body therefore must replenish the total free glucose concentration about nine times a day; nevertheless, the concentration in blood remains remarkably constant. That said, it becomes rather clear why higher or lower levels of blood sugar are associated with the onset of clinical signs and symptoms, which can progress to life threatening conditions if undetected or left untreated.

Not yet classified as a disease state, the third diabetic subgroup nevertheless represents a well-described entity that only recently has been targeted as at-risk for transient hyperglycemic episodes. This subgroup includes individuals with impaired glucose metabolism (i.e., impaired glucose tolerance, “IGT”, insulin resistance, or impaired fasting glucose, “IFG”). These individuals have blood glucose levels that are higher than normal but not high enough meet the diagnostic criteria typically set for diabetes. About 20 million people in the U.S. have IGT, according to the National Health and Nutritional Examination Survey III, and they are at higher risk both for diabetes (as few as 1 to as many as 10 of every 100 persons with IGT is expected to develop full blown diabetes every year) and the complications associated with chronic hyperglycemia. Similarly, a variety of other intercurrent illnesses or pathological conditions can impair glucose homeostatic mechanisms thereby predisposing hyperglycemia and its consequences.

Based on frequency, diabetes has now become the most widespread human metabolic disorder. More than sixteen million Americans (both adults and children) already have some form of diabetes, while as many as five million of these are not yet aware that they have diabetes. Based on current projections, approximately 200,000 Americans die annually as a direct consequence of diabetes and its complications. Demographically, African Americans, Hispanics, Asians and Native Americans are known to have a higher rate of developing diabetes during their lifetime. The scale of the problem that diabetes poses to world health is still widely under-recognized. Estimates predict that if current trends continue the number of persons with diabetes will more than double, from 140 million to 300 million in the next 25 years. Demographically, this means that the greater proportion of the increase is likely to occur in developing countries.

In keeping with its chronic nature, diabetes has potential long-term complications that can affect the kidneys, eyes, heart, blood vessels and nerves. There is good evidence that early and timely preventive strategies and interventions can reduce subsequent morbidity and mortality. From an epidemiologic vantage point, this necessarily embraces all forms of prevention targeted at reducing either the risk of disease onset or improving the outcomes in those manifesting either preclinical or clinical evidence of the disease. Examples include self-administered glucose testing and the adoption of improved menu planning and exercise regimens to mitigate known risk factors.

Identifying and understanding the risk factors associated with diabetes is invaluable for the development and evaluation of effective intervention strategies. Most of the excess morbidity and mortality is related to the chronic complications of the disease rather than to the acute problems that accompanies high or low blood sugars. The complications of diabetes can be divided into three major groups. First and foremost are the microvascular sequellae, which comprise both retinopathy and nephropathy. The second group encompasses the neurological sequellae, which may have a microvascular component with or without disturbed neural function. The third, macrovascular, includes diseases of the large vessels supplying the legs (lower extremity arterial disease), the heart (which typically encompasses coronary artery or major arterial disease) and the brain (cerebrovascular disease). Mortality is of course the severest complication of all and is dramatically increased in both Type 1 and Type 2 diabetes.

The cost of caring for patients with diabetes is considerably higher than for those without the disease. The per capita costs have been estimated at 3 to 4 times those of the non-diabetic population. Most of this increased cost is due to the complications, particularly those requiring hospitalizations. Seventy-five percent of hospitalizations are due to cardiovascular complications. Microvascular complications, including neuropathy, have also been linked to disease duration and blood sugar level. These observations are consistent with the Diabetes Control and Complications Trial (See, DCCT Research Group, N. Engl. J. Med. 329:977-986 (1993), which established that the maintenance of good glycemic control is of major benefit in preventing the development and progression of these types of diabetic microvascular and neuropathic disease.

Lacking normal regulatory mechanisms, diabetics are encouraged to strive for optimal control through a modulated life style approach that focuses on dietary control, exercise, and glucose self-testing with the timely administration of insulin or oral hypoglycemic medications. Invasive forms of self-testing are painful and fraught with a multitude of psycho-social hurdles, and are resisted by most diabetics. Alternatives to the currently available invasive blood glucose testing are highly desirable.

Conventional approaches seek to reduce or eliminate the skin trauma, pain, and blood waste associated with traditional invasive glucose monitoring technologies. In general, noninvasive optical blood glucose monitoring requires no samples and involves external irradiation with electromagnetic radiation and measurement of the resulting optical flux. Glucose levels are derived from the spectral information following comparison to reference spectra for glucose and background interferants, reference calibrants, and/or application of advanced signal processing mathematical algorithms. Candidate radiation-based technologies include: 1) mid-infrared radiation (MIR) spectroscopy, 2) near-infrared radiation (NIR) spectroscopy, 3) far-infrared radiation (FIR) spectroscopy, 4) radio wave impedance, 5) photoacoustic spectroscopy and 6) Raman spectroscopy. Each of these methods uses optical sensors, and relies on the premise that the absorption pattern of infrared light (700-3000 nm) can be quantitatively related to the glucose concentration. Other substances such as water, protein, and hemoglobin are known to absorb infrared light at these wavelengths and easily obscure the relatively weak glucose signal.

Other approaches are based on microvascular changes in the retina, acoustical impedance, NMR spectroscopy and optical hydrogels that quantify glucose levels in tear fluid. While putatively non-invasive, these technologies have yet to be demonstrated as viable in clinical testing.

Nearly noninvasive techniques tend to rely on interstitial fluid extraction from skin. This can be accomplished using permeability enhancers, sweat inducers, and/or suction devices with or without the application of electrical current. One device recently approved by the FDA relies on reverse iontophoresis, utilizing an electrical current applied to the skin. The current pulls out salt, which carries water, which in turn carries glucose. The glucose concentration of this extracted fluid is measured and is proportionate to that of blood. This technology, in keeping with its nearly noninvasive description, is commonly associated with some discomfort and requires at least twice daily calibrations against conventional blood glucose measurements (e.g. invasive lancing).

Other nearly noninvasive blood glucose monitoring techniques similarly involve transcutaneous harvesting for interstitial fluid measurement. Other technologies for disrupting the skin barrier to obtain interstitial fluid include: 1) dissolution with chemicals; 2) microporation with a laser; 3) penetration with a thin needle; and/or 4) suction with a pump. Minimally invasive blood glucose monitoring can also involve the insertion of an indwelling glucose monitor under the skin to measure the interstitial fluid glucose concentration. These monitors typically rely on optical or enzymatic sensor. Technologically innovative, these in situ sensors have had limited success. Implantable glucose oxidase sensors have been limited by local factors causing unstable signal output, whereas optical sensors must overcome signal obfuscation by blood constituents as well as interference by substances with absorption spectra similar to glucose. Moreover, inflammation associated with subcutaneous monitoring may contribute to systematic errors requiring repositioning, recalibration or replacement, and more research is needed to evaluate the effects of variable local inflammation at the sensor implantation site on glucose concentration and transit time.

Interstitial fluid glucose concentrations have previously been shown to be similar to simultaneously measured fixed or fluctuating blood glucose concentrations (Bantle et al., Journal of Laboratory and Clinical Medicine 130:436-441, 1997; Sternberg et al., Diabetes Care 18:1266-1269, 1995). Such studies helped validate noninvasive/minimally invasive technologies for blood glucose monitoring, insofar as many of these technologies measure glucose in blood as well as interstitial fluid.

Given that many of the complications known to be associated with diabetes can be decreased or avoided altogether with proper blood glucose control and the maintenance of a healthy lifestyle, efforts at developing a non-invasive self-monitoring glucose device continue. The challenge is broader than attenuating anticipated meal or exercise induced diurnal fluctuations via glucose testing and appropriate self-medication. Many factors predispose metabolic lability in diabetes, including intercurrent illness, stress, anxiety, pregnancy, or even other medications. The difficulties associated with non-invasive glucometry can be further compounded by the demographic, medical and physiological distinctiveness presented by each potential user of the device. Alone or in combination, these factors can contribute significant alterations in glucose uptake and/or insulin sensitivity. Uncertainty over the potential for large and sometimes rapid glucose fluctuations can amplify the anxiety and discomfort of patients and their parents/families when faced with real questions as to the direction and rate of change of their glucose levels.

Facilitating the ease and convenience of glucose self-assessment would extend the comfort and dedicated commitment of diabetics for knowledgably and actively participating in their own disease management. The ability to obtain timely information about glucose levels and glycemic trends contributes to consistently improved glycemic control by permitting rapid and accurate interventional management.

A noninvasive glucose monitor that is portable, simple and rapid to use, and that provides accurate clinical information is greatly to be desired. In particular, the ability to derive primary and secondary order information regarding real time, dynamic glucose metabolism (such as the direction and rate of change of bioavailable glucose distributed within the blood and interstitial fluid space) is highly desirable.

### SUMMARY OF THE INVENTION

The present invention overcomes the problems and disadvantages associated with current strategies and designs and provides devices, compositions and methods for the non-invasive measurement of in vivo glucose levels.

One embodiment of the invention is directed to a portable spectroscopic system for non-invasively determining the level of an analyte in vivo, where the system contains a light source for illuminating a skin surface with one or more wavelengths of electromagnetic radiation, a detector for detecting radiation emanating from the skin surface after illumination, an optical interface containing an aperture that allows for passage of the electromagnetic radiation from the light source to the skin surface, and passage of radiation from the said skin surface to the detector, and a processor for determining the analyte level from detected radiation.

The analyte may be glucose.

The light source may be fluorescent light, visible light, ultraviolet light, or infrared light, or combinations of these sources. One or more wavelengths of light may be selected from the wavelengths between 200 and 2,500 nm. The detector may be, for example, a photodiode or a CCD array, and the radiation emanating from the skin surface after illumination may be fluorescence, ultraviolet, infrared, visible, diffuse reflectance, or Raman scattering, or combinations of these radiations.

In a particular embodiment, the analyzed skin surface is a surface on the skin of an arm, a leg, a neck, a head, or a torso, or a combination of these surfaces.

The system may contain an optical interface containing a tape comprised of a plurality of zones. The zones may contain a zone for calibration of the system, a zone for measurement of radiation emanating from the illuminated surface, and a zone for storage and protection of the system. All or a portion of the optical interface may be disposable. The tape may be contained within a cartridge or housing such that the tape can be advanced revealing an unexposed portion for sequential calibration and measurement.

The system may be battery powered, and may weigh less than 1 kg, either with or without the batteries.

Also provided are methods for non-invasively determining the in vivo level of an analyte in a patient, by applying the portable spectroscopic system described above to a patient, calibrating the system, obtaining a spectroscopic measurement, and determining the analyte level.

In another embodiment of the invention, there is provided a disposable calibration device for use with a portable optical patient analysis system, containing a housing for an optically transparent medium and a first and a second reservoir suitable for holding the medium. The housing has a window disposed between the first and second reservoirs and a means for urging the optically transparent medium from the first to the second reservoir while passing over the window. Upon contact of the calibration device with the portable patient analysis system the aperture of the patient analysis system is placed in alignment with the window. The optically transparent medium is subdivided into at least a calibration zone and an analysis zone, where the calibration zone is coated with a calibration composition suitable for calibrating the portable optical analysis system, and the analysis zone is uncoated or is coated with a composition suitable for recording data from a patient for analysis. The optically transparent medium optionally may contain a neutral zone coated with a composition that protects the integrity of the aperture or other aspect of the portable patient analysis system.

The medium may be subdivided into repeating areas, where each repeating area comprises at least one calibration zone, at least one analysis zone, and at least one neutral zone. Each repeating area may contain more than one calibration zone or a plurality of calibration zones, which may be the same or different. The device may be in the form of a tape, and the first and second reservoirs may be tape spools. The urging means may operates unidirectionally, permitting transfer of the tape between the tape spools in one direction only. The urging means may be a thumbwheel or lever for manual advancement of the tape, or may comprise a gear or system of gears that functionally couple with an electromechanical winding means, for example on the hand held device described above, permitting transfer or advancement of the medium from the first reservoir to the second reservoir.

Other embodiments and advantages of the invention are set forth, in part, in the following description and, in part, may be obvious from this description, or may be learned from the practice of the invention.

### DESCRIPTION OF THE DRAWINGS

FIG. 1 depicts one embodiment of the system comprising a hand-held instrument.

FIG. 2 depicts one embodiment of a calibration tape.

FIG. 3 depicts an embodiment of a hand-held instrument for fingertip measurement.

FIG. 4 depicts another embodiment of the calibration tape within a cartridge device.

FIG. 5 depicts another embodiment of the instrument comprising the flashlight model.

FIG. 6 depicts another embodiment of the instrument comprising a finger grip device.

### DESCRIPTION OF THE INVENTION

The inventions provides new devices and device components for use in methods of non-invasive tissue monitoring. In particular, the devices and device components are useful for non-invasive monitoring of glucose levels, although the skilled artisan will recognize that the devices and components may be used in a wide variety of non-invasive monitoring methods for many different analytes.

One embodiment of the invention is directed to a device that is compact, preferably hand-held, for monitoring physiologic or metabolic events. Preferably, the device can be used by diabetic patients to routinely and repeatedly measure their own glucose levels without the need to extract and sample portions of bodily fluids or cells, and without the aid of medically-trained personnel. The device can be used in any setting, for example in the patient's home or office, and provides robust and accurate data that the patient can reliably use for disease self-management such as, for example, to decide on appropriate dietary or exercise changes, and/or the administration of insulin or other drugs and biologically active materials.

Component of the invention include an optical interface such as, for example, a strip of film or tape that is positioned between the skin surface and the optics of the device. Preferably, the strip contains a plurality of regions that variously permit, for example, the machine to be calibrated, the collection of data for monitoring physiologic or metabolic status, and optionally that protect the integrity of the device when turned off or otherwise in stand-by mode. This component is particularly suited for use with the hand-held monitor of the invention.

A. Hand-Held Instrument

The hand-held device of the invention overcomes the problems and disadvantages of prior monitoring apparatus by providing a non-invasive and direct measurement of physiologic status, preferably glucose levels, based on the detection of fluorescent radiation emitted or emanating from a patient in response to excitatory energy. The energy is applied directly to the outer surface of the patient's skin, obviating the need for obtaining bodily fluids. The device provides an exciting pulse of energy, a means for detecting emitted or emanating energy, a calculating means that converts detected data into meaningful information that is understood by the operator, and a display for presenting the calculated data to the operator. The device preferably contains an optical interface and the interface is preferably a strip of tape or film.

Optical Excitation of the Skin

For transmitting the excitatory energy to the patient's skin, the device contains at least one or a plurality of each of a light source, optics and a filter system or monochromator. Together these components form the optical interface of the device. In one embodiment of the invention, the light projected is broadband and passes through a filter system such that discrete wavelengths illuminate the skin. In other embodiments, the light source may be tunable emitting only specific wavelengths.

In one embodiment, the radiation source is configured to emit excitation radiation at a plurality of different wavelengths. Preferably, the radiation may be ultraviolet light, visible light, fluorescent light, infrared light, or combinations of these. The radiation may have a wavelength or wavelengths between 200 and 2500 nm. Suitable radiation sources are disclosed in U.S. patent application Ser. Nos. 09/287,486 and 09/785,547, and PCT Application No. WO 01/60248, the disclosures of which are hereby incorporated by reference in their entireties. Suitable ultraviolet light sources include a continuous mercury lamp, a pulsed or continuous xenon flash lamp, or a suitable laser. Useful lasers include, but are not limited to, nitrogen lasers, doubled OPO (tunable laser) and tripled Nd YAG pump devices. Useful pulsed sources include a 2-channel lock-in amplifier or a gated CCD. The source output may be filtered to restrict illumination to within excitation bands of interest. Intensity and pulse width, if applicable, may be set at a level that minimizes exposure while optimizing signal-to-noise considerations. The sample may be irradiated with two or more short (e.g., femtosecond) pulses of multi photon light having a wavelength two or more times longer than the wavelength of interest, such that the radiation penetrates to a different degree or depth. The excitation radiation is projected from an optical window on the end of the instrument.

In one embodiment, the device incorporates the a xenon flash lamp, providing illuminating light over a broad spectrum with wavelengths ranging from approximately 150 nanometers (nm) to approximately 2000 nm. Consistent with the design and intent of the present invention, the flash apparatus can be powered via a coupled battery source, where the charging circuit is coupled to the battery power source and the charging circuit has an oscillating circuit, a voltage step-up transformer and a storage capacitor.

In another embodiment, a microprocessor timing circuit replaces the oscillating circuit. A neon or LED ready light or other visual indicator, or a sound indicator, may be connected in series or parallel across a flash storage capacitor to inform the operator (which can be a patient, a nurse, a physician or other clinical care worker), when sufficient charge is stored in capacitor, e.g. +300 volts, to accomplish a flash illumination within the flash tube. When the capacitor charge voltage reaches substantially full charge, a sensor momentarily conducts, stopping the oscillation in the charging circuit. Triggering is the initiation of an electrical discharge in the gas contained in the flash lamp. Triggering typically begins with a spark streamer that crosses the gap between the electrodes and creates a conductive path between them. The voltage drop across this path is generally less than the voltage supplied by the external circuit, so current will begin to flow through the lamp. The trigger capacitor is charged by current flow through charging transformer secondary winding at the same time and in similar manner as the storage capacitor. When activated, the trigger capacitor then discharges through the primary winding of the transformer, inducing a high voltage pulse of about 500 volts to 6 kilovolts in the secondary winding. This causes ionization of the gas in the flash discharge tube resulting in the storage capacitor discharging through the flash tube, producing flash illumination. The output of the flash lamp (such as wavelength, intensity, flash duration) can be optimized or varied by changing the gas mixture, fill pressure, tube size and shape of the flash lamp.

In a preferred embodiment, the light is flashed by the operation of a one-touch button, operable by the device user. Depression of the button initiates a flash or series of flashes with an on/off switch initiating a charging cycle to charge a storage capacitor to provide energy for operation of the flash tube. The device or parts of the device maybe monitored or controlled by a microprocessor.

Detection of Radiation from the Target

Detection of the radiation emitted by or emanating from the target (e.g. the patient's skin surface) at the optical interface can be detected by means well known in the art. Suitable detectors are described in U.S. patent application Ser. Nos. 09/287,486 and 09/785,547, and PCT application WO 01/60248. Briefly, the detector may be a photodiode or CCD array for detection of fluorescence emitted back from the patient's skin after excitation. Preferably, emitted radiation enters the instrument back through the same optical window as the exciting radiation. The detected radiation may be fluorescence, ultraviolet, infrared, diffuse reflectance, Raman scattering, and combinations of these.

For glucose monitoring in particular, excitation and detection of fluorescence preferably provides a broad spectrum of light for adjunctive spectroscopic analysis incorporating diffuse reflectance, and/or additional techniques including but not limited to ultraviolet (UV), visible, infrared (IR) which includes near infrared (NIR), mid infrared (MIR) and far infrared (FIR), visible light absorbance, Raman, microwave and/or combinations of these spectral regions.

When the radiation source is configured to emit excitation radiation at a plurality of different wavelengths, the radiation detector is configured to synchronously scan radiation emitted by the target with the excitation radiation (e.g. an excitation-emission map, in which the excitation-emission pairs for fluorescence are represented in a three dimensional array with the X and Y axes representing excitation and emission wavelengths respectively with the Z axis corresponding to the fluorescence intensity returned at excitation wavelength X and emission wavelength Y).

Combination of the Radiation Source and Detector in a Simple Hand-Held Device

The device is preferably convenient and easy to use such as a device that can be entirely held in the hand or held by a handle, and sufficiently light weight. Accordingly, in one embodiment the device resembles a flashlight that contains both the excitation source and the detector, where the device can be placed against the skin in an area where there is a flat surface, such as the inner arm, forearm or thigh to allow optimum skin contact.

Depression of an activation button or similar ergonomic device triggers illumination of the light source, typically followed by detection of emitted radiation. In devices containing the calibration tape component described in more detail herein, depression of the activation button also may cause the automatic advancement of the calibration tape or film to that portion of the tape or film that permits irradiation of the target and subsequent detection of the emitted radiation.

The device also contains a computing device, such as a microprocessor, that is functionally coupled to the detector and that performs the necessary calculations to convert the raw detection data into cognizable information for the user. Thus, the computing device receives the detection data from the detector and applies algorithms that are known in the art, or that can be designed using methods that are known in the art, to the data, and calculates, preferably, an in vivo glucose level.

The resulting information can be displayed to the patient in a wide variety of ways that are known in the art. For example, the device may include an LCD read-out that conveniently displays glucose information similar to the information currently provided by traditional invasive blood glucose monitors. In another embodiment, a series of lights may indicate whether the glucose is in acceptable range (green), is high or low (yellow), or is dangerous (red).

The device may also optionally contain a memory chip that stores data that are collected over a period of time, and optionally may further contain a port to down load the data to any other device as may be desired. Suitable memory chips and ports are known in the art. The device may also include rechargeable batteries for convenient use.

In a particular embodiment, the device resembles a large bar of soap with the intended site of radiation application being the inter digital web between the index and middle fingers. The device is designed so that it fits easily into the hand. Suitable ergonomic shapes for such devices that are readily gripped by a variety of hand sizes and shapes are well known in the art. The patient's hand grips the device and irradition/detection is activated by pressing a button, most conveniently with the thumb. The optical interface is contained in a molded finger that projects smoothly from the device and fits snugly between the fingers. The molded finger optionally contains the a disposable calibration tape described in more detail herein that assures the integrity of the measurement for each use.

In a further embodiment, the device resembles a large bar of soap that is most conveniently used while resting on a flat surface. The intended site of application in this embodiment is a fingertip. The patient places a fingertip on a touch pad and activates the device either by 1) pressing on a touch pad or similar ergonomic device, activating the measurement when a predetermined pressure is reached or 2) pressing a button on the side of the device with the thumb. When a touch pad is used, the pad lies above a suitable pressure sensor that is calibrated to activate illumination once the applied pressure reaches a certain threshold. This threshold may be devised so as to activate the device once the patient makes a sufficiently robust contact with the device that permits accurate and reproducible measurements to be made. By way of example, if the device is triggered when contact is made over an insufficient area, the accurate measurement of data may be confounded by the absence of any data from those areas where no contact is made, leading to unreliable results. Use of a suitable pressure sensor eliminates this possibility.

Similarly, even though contact may be made over a sufficient area to avoid unwanted “blank” readings, insufficient pressure of contact may lead to an inconsistency in the nature or quality of the of contact over the irradiation/detection area, again leading to possibly unreliable results. Again, use of a suitable pressure sensor that triggers irradiation/detection only when a desired threshold is attained eliminates this problem.

Extreme variation in the temperature of the hand or in hydration may lead to unreliable results. For example, very cold hands may also be very dry, or may have less flexible skin, leading to non-optimal contact with the device. Accordingly, the device also may contain a temperature and/or humidity sensor that ensures that detection occurs only when the contacted tissue is at an appropriate temperature.

To improve the quality of the contact between the skin of the patient and the optical interface of the device, an interface medium can be used. Compositions suitable for use as an interface medium are known in the art and are also described in U.S. patent application Ser. No. 09/704,421 and PCT/US00/30306, which are hereby incorporated by reference in their entirety.

The interface medium may comprise a viscous composition, such as a liquid, paste or gel, having an index of refraction that matches or approximates the indices of both the tissue surface and the optical interface of the device. Preferably, the index of refraction is between 1.1 and 2.0, more preferably between 1.2 and 1.8 and even more preferably 1.4. The medium is preferably clear in the visible spectral region of 400-700 nm, more preferably in the region of 270-500 nm, and typically is pH buffered, and safe for long term application to skin surfaces. The medium is non-toxic in the concentrations used for measurement.

Use of the medium allows for improved tissue contact, even in the presence of dry skin, scaling skin, or air pockets due to skin texture, or other irregularities such as pits in the nail bed. The medium also minimizes data variability based on skin differences such as pigmentation, thickness, blood flow, and like physiological variables. Medium is preferably slippery, allowing for reduced friction and mechanical stress between the skin and device. Further, accuracy and reproducibility are enhanced by providing improved thermal contact between the skin and the device. This thermal buffering stabilizes and increases the thermal stability of the interface. Stabilization or control of the local environment augments and improves data acquisition.

Interface medium is preferably water soluble for ease of application and removal and may be non-staining, but in some applications may be water insoluble. In one embodiment, the medium comprise as the principal component an optically inactive ingredient, that is, substantially inert and substantially transparent to allow the transfer of light with no more than negligible interference, for example, glycerin, polyethylene glycol such as, for example, most any PEG such as PEG-200, PEG-400 or PEG-600, polypropylene glycol, phosphate or combinations of these ingredients, and one or more buffers and/or wetting agents. Additional secondary components include PEG-150 stearate or distearate, glyceral stearate, cetyl alcohol or combinations thereof. Concentrations for the secondary components range from 0.01% to 20%, preferably 0.1% to 10%, and more preferably 1% to 5%, and even more preferably 2%.

In a preferred form, the device is manufactured expressly to suit the needs of diabetics, and incorporates explicit ergonomic features to confer convenience and ease of use to a wide population of diabetics. Hence, in one embodiment, the device is sufficiently portable to permit easy manual handling without preference to handedness. The device may have an LCD-type display (backlit, monochromatic or color) with adjustable font sets to ensure readability for users with compromised visual acuity. The device may also have a plurality of buttons that permit patient input of information and the setting up personalized logs, diaries, profiles, or programs that can be scaled to accommodate visual and/or sensory impairments. To facilitate ease of inputting and reading, the monitor may have direct cable or wireless connectivity options to facilitate data transfer to a personal computer. The skilled artisan will recognize that suitable software can enhance data organization for storage and/or exchange with preferred medical caregivers. The device may have data entry options including over ride preprogrammed buttons and software based text entry.

In another embodiment, the portable device accepts a plug-in voice adapter to enable visually impaired users to monitor their glucose levels independently by using a meter with a synthetic voice. This voice-adapted meter provides audio readings of the messages that appear on the meter display panel. Another provision of the audio adapter is that it can be programmed to read medication bar codes (e.g. those found on insulin vials). Calibration coding information can be directly entered manually or via preprogrammed strips or chips into the voice adapter.

The device advantageously weighs less than 1 kg and preferably less than 1/2 kg, and possesses a convenient shape for holding and carrying to maximize portability.

Applications for the Hand-Held Instrument for Diabetics

The device is of particular value in settings characterized by extreme glucose lability where loss of insulin-to-glucose homeostasis can become life threatening as with severe dehydration, extreme exertion, or inter current infection.

The ability to accurately and conveniently measure glucose levels repeatedly and non-invasively at numerous intervals is particularly valuable because glucose levels can vary tremendously and often unpredictably over time. This lability is especially challenging for diabetics, who lack the control mechanisms for maintaining glucose homeostasis. Moreover, the absolute level of a single glucose measurement is considerably less useful from a therapeutic vantage point than an understanding of the direction and rate of change of the glucose levels (such as rising or falling and the momentum associated with the change). Eliciting this type of information from one or a plurality of closely spaced serial measurements provides a database that is rich enough to permit the patient to make decisions regarding diet or insulin dosing that are based on their current or actual situation, as distinguished from anticipated medication requirements.

In an alternative form of the invention, the device provides for independent, isolated glucose level measurements as a means of assessing current efforts at maintaining glycemic control as described substantially in U.S. patent applications Ser. Nos. 09/287,486 and 09/785,547, which are incorporated herein by reference in their entireties. In a further alternative form of the invention, the device could be functionally associated with a radio telemetry transmitter (see IEEE 802.11), blue tooth wireless or other standard for wirelessly transmitting data to a family member or healthcare professional for review, analysis and/or intervention. This includes placing the analysis and transmissions components into a device amenable for use in a Palm™ or Visor™-type PDA.

B. Optical Coupler

The present invention also provides an optical coupler for connecting the instrument to the skin surface. The coupler comprising an aperture that allows for transmission of electromagnetic radiation from the light source to the skin surface. The same or a different aperture may allow for transmission of radiation from the skin to the detector. The coupler may further include calibration components such as, for example, filters and reflectors that select for specific wavelengths or groups of wavelengths. As such, these filters and/or reflectors select for specific type of skin heterogeneity, pressure, moisture content, color, and/or other physiologic parameters. In particular, the calibration element is suitable for use with the hand-held devices, but may also be used with other non-invasive measurement devices that employ spectroscopic or spectrophotometric methodology.

The device may further comprise a reliable method of calibration to ensure accuracy of measurement. This is particularly important for portable devices that are intended to be used in a wide variety of persons with little to no training, such as portable glucose monitors for home use. As the skilled artisan will appreciate, the variation in the physical environments or situations under which a portable unit will be used means that data collection should be calibrated under the same conditions as the intended measurements.

The present invention provides a disposable calibration element that may be placed within an optical interface. The interface is a medium such as a strip of tape or film (hereinafter a “tape,” though the skilled artisan will recognize that the physical format of the calibration medium is not limited to tape). A reservoir of tape is provided from which the tape can be advanced as it is used, to be collected in a second reservoir. For example, the tape may be wound on a spool or cartridge. As the tape is used, it is collected in another reservoir, for example another spool or cartridge. Advantageously, the tape is arranged between the two reservoirs in such a fashion that it can be advanced in only one direction, preventing reuse of the previously exposed or contaminated tape, which could lead to degradation of instrument performance. For example, spools may be arranged in a similar fashion to disposable cameras, which allow advance of the film in a single direction to avoid accidental multiple exposures. In one embodiment, this can be achieved by providing an urging means for advancing the medium between the first and second reservoir. For example, a sprocket on the spool on which the calibration tape is wound may be used such that the spool can turn only in one direction. Thus, as the tape advances, small teeth penetrate the surface of the tape aiding advancement and barring the tape from being used again.

The tape itself can be sufficiently transparent to the wavelengths of radiation used in the optical device that it permits transmission of both excitatory radiation from the device, and emitted radiation from the patient. Suitable tapes are known in the art for a wide variety of wavelengths and ranges of wavelengths of excitatory and emitted radiation. The tape advantageously is resistant to stretching so that the thickness of the tape at the optical interface is relatively constant. However, the tape also advantageously is flexible, since it is intended that the tape will contact the patient's skin when the device is used. The non-uniform surface of the patient's skin therefore means that the tape preferable is somewhat flexible. However, the skilled artisan will recognize that, in a device that uses an optical interface of relatively small surface area, the non-uniformity in the patient's skin surface is less important, and the tape, may correspondingly be less flexible.

The tape may be uniform or divided into repeating units. Each repeating unit contains a plurality of zones, each with a predetermined purpose associated with the particular use or analyte to be detected. The first zone may be a “calibration” zone. In this zone tape carries a coating of a known amount of a calibrant composition. Thus, for example, where the tape is to be used in a portable device for measuring a patient's glucose levels by excitation and measurement of fluorescence, the tape contains a coating that fluoresces within the expected detection range for a typical patient. For example, the device can measure the wavelength and amplitude of the radiation emitted by the calibration zone upon irradiation. The device can use the result obtained from the calibration zone to apply suitable corrections to actual data obtained from a patient, thereby increasing the accuracy of those data and/or nullifying heterogeneity between different surfaces. Alternatively, if the data obtained from the calibration zone vary too greatly from the expected range, this can indicate either that the device is malfunctioning, or is being used in an unsuitable environment. The calibrant coating is advantageously placed on the surface facing the optical window, and typically is opalescent.

Alternatively, a series of two or more calibrant zones can be used, each containing differing calibrant coatings. This permits still more accurate calibration of the device over a wide detection range, and/or in a wide variety of environments.

When the tape is advanced, either manually (for example by a thumbwheel or lever such as those used in manual cameras) or automatically (for example using an electromechanical means, such as a simple auto winder device akin to those used in some cameras), the calibration zone is followed by an analysis zone. This zone is sufficiently optically clear to permit transmission of exciting and emitted radiation from the device. Alternatively, the calibration zone may be a filter that allows for the transmission, detection or both, of only desired wavelengths. This can nullify errors, and eliminate background. For use of an electromechanical means of advancing the medium, the device may contain a gear or system of gears that interact with a winding means that is external to the calibration device, for example on the portable analysis system.

After advancement of the tape from the analysis zone, the tape optionally may contain a neutral zone, which covers the optical interface or lens of the instrument and protects it when the device is not in use. The neutral zone typically is opaque to the wavelengths of radiation used in the device. Advantageously, the tape is self advancing so that, for example, when the device is turned on or placed in the necessary mode for data collection, the tape advances to the calibration zone and the device calibrates itself. The tape then advances to the analytical zone where the device makes a measurement of the parameters desired, followed by advance of the tape to the neutral zone where it protects the optical interface of the instrument until the next reading is taken. When turned on again the tape automatically advances to the calibration zone before making the next measurement.

By designing the calibration system as described the integrity of each measurement may be assured, as well as, providing a means of protection for the optical interface or lens system. Furthermore, by designing the calibration tape to automatically wind on a spool with each measurement, error due to an uncalibrated reading can be avoided and, in addition, a used tape can easily be removed and a replacement tape easily inserted for uninterrupted use. This design allows for the accurate measurement by a non-expert and reduces the opportunity for operator error.

This disposable calibrant provides a simple and inexpensive means for routine calibration and device standardization prior to each use. This approach also allows for sequential calibration and analysis without requiring instrument disassembly or other disruption of device integrity.

The combination of the hand-held device and calibration means described above overcomes problems and disadvantages associated with current strategies and designs for patient self-monitoring. In particular, the combination is particularly suitable for monitoring of glucose levels in diabetics and similarly situated patients, by permitting integration of the data derived from one or serial in vivo glucose level measurements obtained non-invasively using an optical spectroscopic technology over a short interval. This provides meaningful information about glycemic levels, permits trend analysis, and provides anticipatory management information about glycemic slope (such as direction and rate of change of systemic glucose levels). The present invention also introduces a new system and method for device calibration which maintains the integrity of data measurement at every use. As described conceived herein the invention provides new, unexpected and superior results.

The following examples are offered to illustrate embodiments of the present invention, but should not be viewed as limiting the scope of the invention.

### EXAMPLES

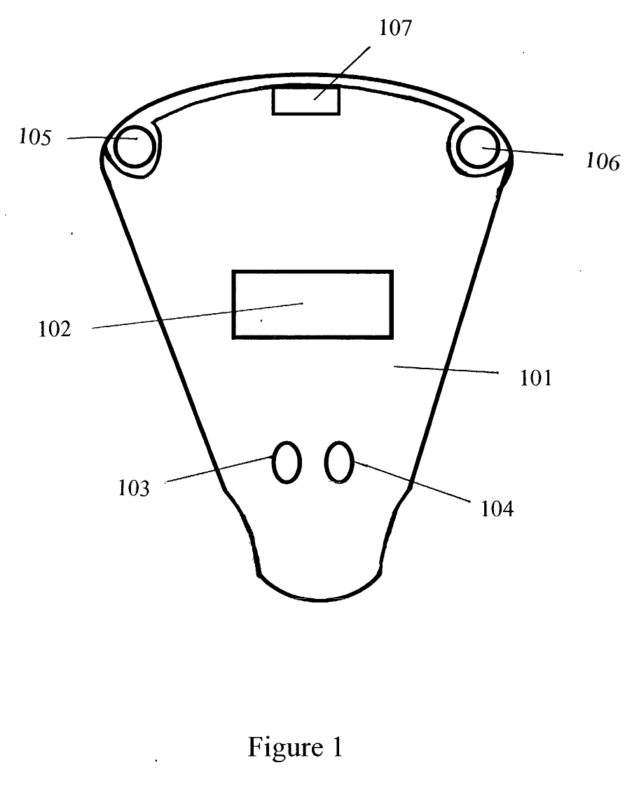

One embodiment of the invention is depicted in FIG. 1, which shows a handheld embodiment of the instrument. Instrument body 101 contains LCD 102 that displays a glucose reading or calibration reading. Buttons 103 and 104 advance the calibration tape dispenser from sprocket 105 to sprocket 106, past window 107 from which readings of skin surfaces are obtained. This device is broadly useful for a wide variety of skin surfaces across the body including skin surfaces of an arm, a leg, the neck, the head, and the torso.

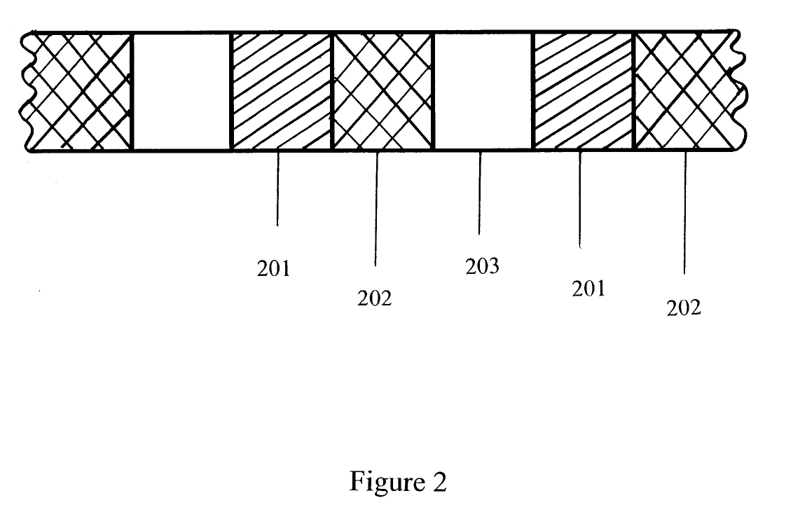

Shown in FIG. 2 is an optical coupler for use in the instrument of FIG. 1 containing zone 201 for instrument calibration, clear analysis zone 202 for obtaining a reading, and protective neutral zone 203 for when the instrument is not in use. The calibration tape sits inside the instrument covering the optical window.

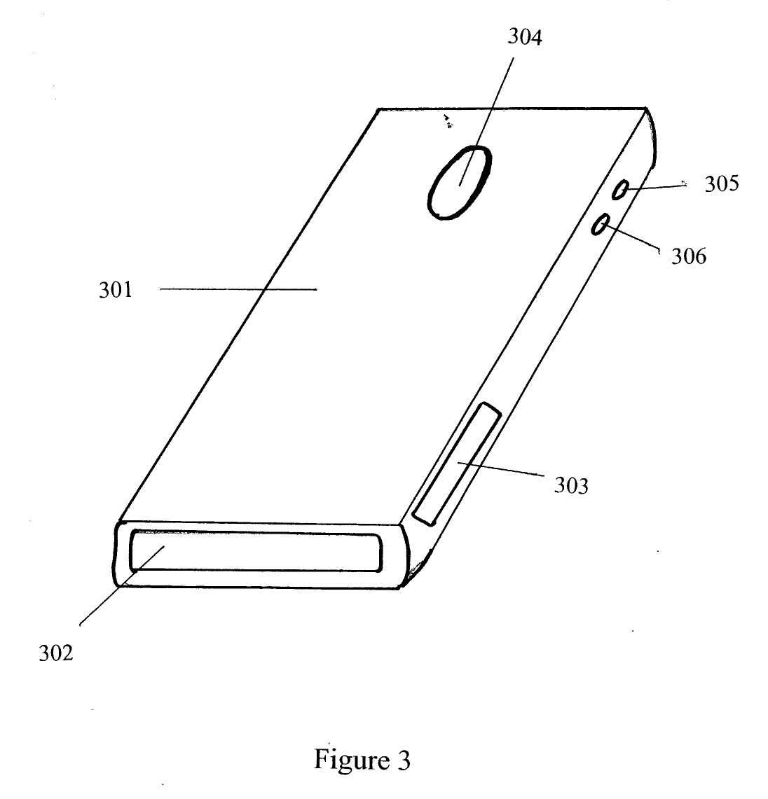

Another embodiment of the instrument is shown in FIG. 3. The body of instrument 301 possesses LCD readout window 304 on the top with two buttons 305 and 306 to one side for advancing and retarding movement of calibration tape (not shown) past window 304. Alternatively, one button may be an activator switch and the other for selection of available modes and wavelengths.

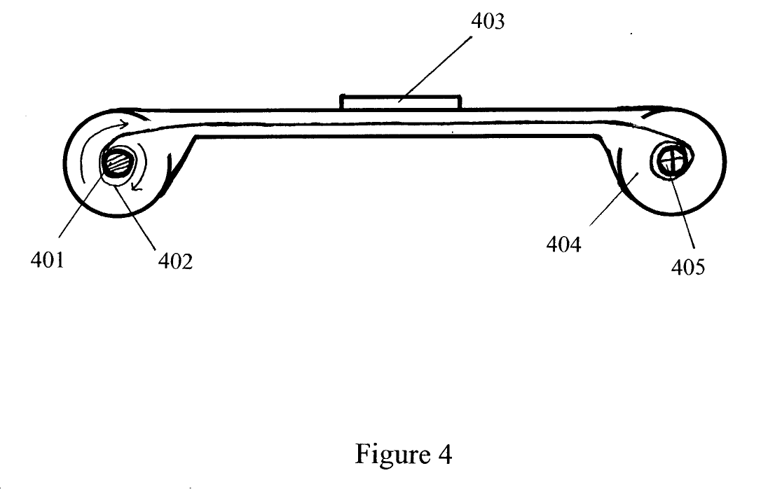

Another embodiment of the invention, a tape cartridge which can be used with the instrument of FIG. 3, is shown in FIG. 4. The cartridge comprises sprocket 401 onto which is wound unexposed tape 402. Upon advancement of the tape through the cartridge, tape becomes exposed at window 403. Exposed tape 404 then winds around sprocket 405. Once all tape has been exposed, the cartridge can be replaced.

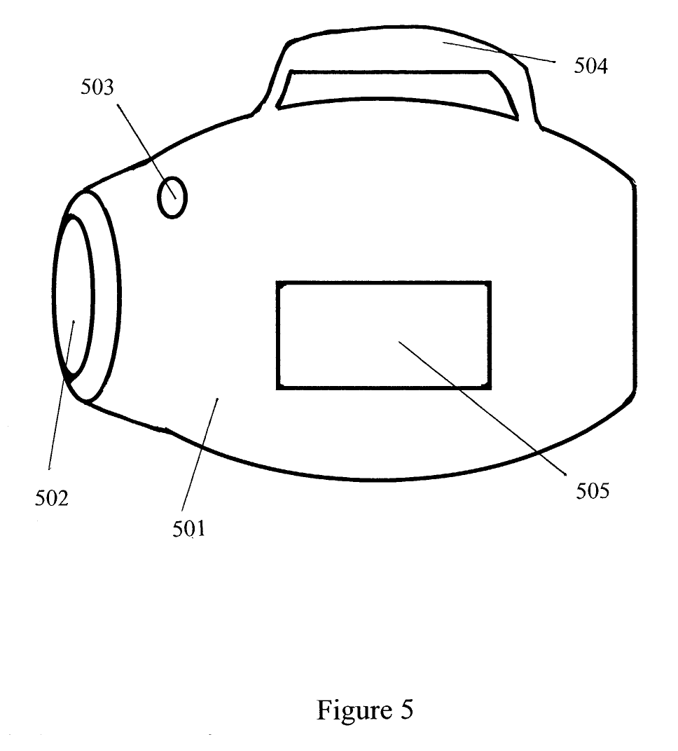

FIG. 5 depicts the flashlight model of the instrument. Housing 501 contains optical coupler 502 which contacts a skin surface. Housing 501 further contains selector switch 503 for selecting a particular wavelength or set of wavelengths, and molded handle 504. LCD readout 505 displays the glucose reading of the patient.

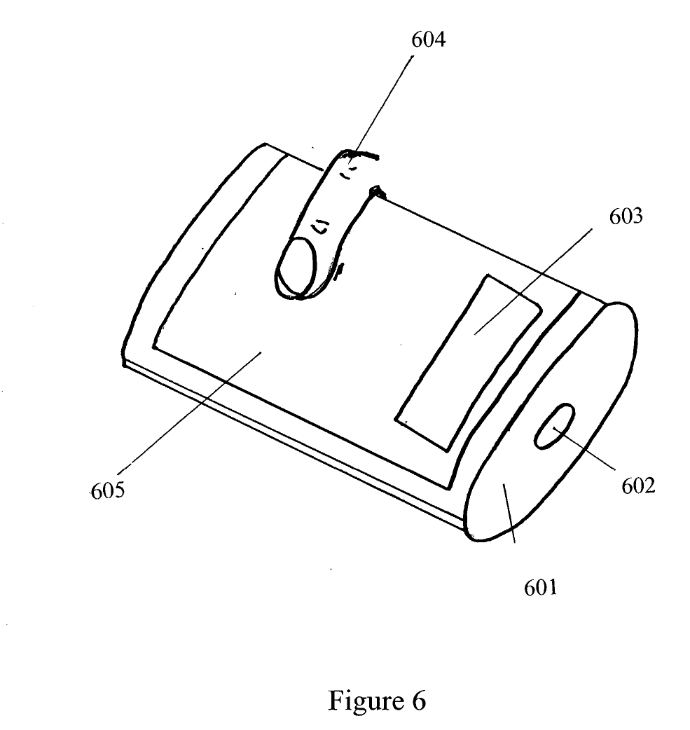

FIG. 6 depicts the top surface of the touch pad instrument whereby finger 604 is placed over a window (not shown) when the device is gripped with the hand. On one side 601 is placed activator button 602 and on top, next to the finger window is LCD readout 603. Top surface contains cover 605 which can be opened for the insertion of the calibration tape and batteries.

Other embodiments and uses of the invention will be apparent to those skilled in the art from consideration of the specification and practice of the invention disclosed herein. All references cited herein for any reason, including all U.S. and foreign patents and patent applications, are specifically and entirely incorporated by reference. It is intended that the specification and examples be considered exemplary only, with the true scope and spirit of the invention indicated by the following claims.

1. A portable spectroscopic system for non-invasively determining the level of an analyte in vivo comprising:
a light source for illuminating a skin surface with one or more wavelengths of electromagnetic radiation;
a detector for detecting radiation emanating from said skin surface after illumination;
an optical interface containing an aperture that allows for passage of said electromagnetic radiation from said light source to said skin surface, and passage of radiation from said skin surface to said detector; and
a processor for determining said analyte level from detected radiation.

2. The system of claim 1 wherein the analyte is glucose.

3. The system of claim 1 wherein the light source is selected from the group consisting of fluorescent light, visible light, ultraviolet light, infrared light, and combinations thereof.

4. The system of claim 1 wherein the skin surface is a surface on the skin of an arm, a leg, a neck, a head, a torso, or a combination thereof.

5. The system of claim 1 wherein the one or more wavelengths are selected from the wavelengths between 200 and 2,500 nm.

6. The system of claim 1 wherein the detector is selected from the group consisting of a photodiode or a CCD array.

7. The system of claim 1 wherein the radiation emanating from the skin surface after illumination is selected from the group consisting of fluorescence, ultraviolet, infrared, visible, diffuse reflectance, Raman scattering, and a combination thereof.

8. The system of claim 1 wherein the optical interface contains a tape comprised of a plurality of zones.

9. The system of claim 8 wherein the tape is contained within a cartridge such that the tape can be advanced revealing an unexposed portion for sequential calibration and measurement.

10. The system of claim 8 wherein the plurality of zones comprises a zone for calibration of the system, a zone for measurement of radiation emanating from the illuminated surface, and a zone for storage and protection of the system.

11. The system of claim 1 wherein all or a portion of the optical interface is disposable.

12. The system of claim 1 which is battery powered.

13. The system of claim 1 which weighs less than 1 kg.

14. A method for non-invasively determining the in vivo level of an analyte in a patient comprising:
applying the portable spectroscopic system of claim 1 to said patient;
calibrating the system;
obtaining a spectroscopic measurement; and
determining the analyte level.

15. A disposable calibration device for use with a portable optical patient analysis system comprising
a housing comprising an optically transparent medium and a first and a second reservoir suitable for holding said medium,
wherein said housing comprises a window disposed between said first and second reservoir and a means for urging said optically transparent medium from said first reservoir to said second reservoir while passing over said window, wherein upon contact of said calibration device with said portable patient analysis system the light transmitting aperture of said patient analysis system is placed in alignment with said window,
wherein said optically transparent medium is subdivided into at least a calibration zone and an analysis zone,
wherein said calibration zone is coated with a calibration composition suitable for calibrating said portable optical analysis system, and said analysis zone is uncoated or is coated with a composition suitable for recording data from a patient for analysis.

16. The device of claim 15 wherein the optically transparent medium further comprises a neutral zone coated with a composition that protects the integrity of the light transmitting aperture of said portable patient analysis system.

17. The device of claim 16 wherein the medium is subdivided into repeating areas, wherein each repeating area comprises at least one calibration zone, at least one analysis zone, and at least one neutral zone.

18. The device of claim 17 wherein each repeating area comprises a plurality of calibration zones, which may be the same or different.

19. The device of claim 15 wherein the medium is in the form of a tape.

20. The device of claim 19 wherein the first and said second reservoir are tape spools.

21. The device of claim 20 wherein the urging means operates unidirectionally, permitting transfer of said tape between said tape spools in one direction only.

22. The device of claim 15 wherein the urging means is a thumbwheel or lever.

23. The device of claim 15 wherein the urging comprises a gear or system of gears that functionally couple with an electromechanical winding means, thereby permitting transfer of said medium from said first reservoir to said second reservoir.
